(19) United States
(12) Patent Application Publication
(10) Pub. No.: US 2005/0155099 A1
(43) Pub. Date: Jul. 14, 2005

(54) Oxygen-controlled environment for cell-and tissue culture

(76) Inventors: Barry E. Rothenberg, Del Mar, CA (US); Fusun N. Zeytin, Del Mar, CA (US); Robert D. Fish, Tustin, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/014,148

(22) Filed: Dec. 15, 2004

Related U.S. Application Data
(60) Provisional application No. 60/530,429, filed on Dec. 16, 2003.
(60) Provisional application No. 60/530,408, filed on Dec. 16, 2003.

(51) Int. Cl. C12N 15/86

(52) U.S. Cl. 800/21; 435/456

(57) ABSTRACT

A primary mammalian cell which may be infected with a pathogen is cultivated under reduced oxygen to improve susceptibility to infection with a pathogen, pathogen propagation, and/or to change or maintain a degree of differentiation of the cell in a predetermined manner. In preferred aspects, the cell is a hepatocyte, optionally infected with HCV, or a blastomere from a single-cell biopsied blastocyst. Such biopsied cell may then be expanded to a population of stem cells under concurrent cultivation of the blastocyst, which is then used for IVF implantation.

This application claims the benefit of U.S. provisional patent applications with the Ser. Nos. 60/530,429 and 60/530,408, both of which were filed Dec. 16, 2003, and both of which are incorporated by reference herein.

### FIELD OF THE INVENTION

The field of the invention is cell and tissue culture, especially as it relates to culture of cells and tissue under a reduced-oxygen atmosphere (i.e., less than 20 vol %).

### BACKGROUND OF THE INVENTION

Stable primary culture of hepatocytes and hepatic tissue is notoriously difficult, and there is currently no indication in the literature that reports successful culture of primary hepatocytes of hepatic tissue. Indeed, all or almost all of the present techniques for stable hepatocyte culture rely on immortalized cells. For example, HepG2 is a hepatoma cell line, while THLE-2 or THLE-3 are hepatocytes immortalized with the SV40 large T antigen.

While such immortalized human liver cells often provide an in vitro model for various diseases and conditions (e.g., pharmacotoxicological studies, or etiology and pathogenesis of human hepatocellular carcinoma), their altered genetic make-up frequently skews transcriptional and/or translational analyses. Moreover, viral infection and propagation of hepatotropic viruses (and especially of the HCV virus) in such hepatocytes is typically unsuccessful due to poorly understood limitations.

Consequently, elaborate measures have been taken to provide an in vitro model that at least in some respects will provide insight into the virus' life cycle. For example, Bartenschlager et al. describe a subgenomic replication unit in HepG2 cells (Science (1999), Vol. 285, pp. 110). Similar approaches have been undertaken using infectious genomes (see e.g., Kolykhalov et al., Science (1998), Vol. 277, pp. 570; M. Yanagi, et al. Proc. Natl. Acad. Sci. (1997), Vol. 94, pp. 8738; M. Yanagi et al., Virology (1998), Vol. 244, pp. 161). Unfortunately, such systems typically provide only limited information (e.g., MS5B inhibition by nucleoside analogs, but not viral assembly and/or viral entry into the cell).

To overcome at least some of these problems, animal models have been developed that allow viral infection (e.g., chimpanzee) of a treatment naïve animal, or that allow other manners of quantification of efficacy of antiviral drugs (e.g., luciferase in transgenic or infected rodents). However, numerous new difficulties arise from almost all animal models, and significant costs as well as concerns regarding transferability of results have negatively impacted the usefulness of such models.

Similarly, expansion of stem cells is often difficult, especially where the initial stem cell population is relatively small. Numerous attempts were performed to improve stem cell culture, but so far, all or almost all of them relied on a relatively large number of initial cells that were then cultivated to expand the population. While embryonic stem cells hold at least potentially great promise for the treatment of numerous diseases, significant ethical debate has arisen, and especially with respect to the source and methods of obtaining such embryonic stem cells. For example, viable mouse embryonic stem cells are typically obtained at the expense of the life of the preimplantation embryo (Evans, et al. Nature 292: 154-159, 1981; Martin, Proc. Natl. Acad. Sci. USA 78: 7634-7638, 1981), or from fetal germ cells extracted from a microdissected embryo (Matsui, et al., Cell 70: 841-847, 1992). Similar work to isolate pluripotent cell lines from various other animals is described elsewhere. (Evans, et al., Theriogenology 33(1): 125-128, 1990; Evans, et al., Theriogenology 33(1): 125-128, 1990; Notarianni, et al., J. Reprod. Fertil. 41(Suppl.): 51-56, 1990; Giles, et al., Mol. Reprod. Dev. 36: 130-138, 1993; Graves, et al., Mol. Reprod. Dev. 36: 424-433, 1993; Sukoyan, et al., Mol. Reprod. Dev. 33: 418-431, 1992; Sukoyan, et al., Mol. Reprod. Dev. 36: 148-158, 1993; Iannaccone, et al., Dev. Biol. 163: 288-292, 1994).

Technically, substantially similar procedures could be performed using human embryos, and recent reports indicate that unused preimplantation embryos from in vitro fertilization procedures (IVF) have been employed for embryonic stem cell production (e.g., in Singapore, or in the United Kingdom) where the embryo was less than 14 days old (Culture conditions were described in Bongso et al., Hum Reprod 4: 706-713, 1989). A further protocol for production of embryonic stem cells is described in U.S. Pat. No. 6,200,806, in which the inventors carefully emphasize the use of non-human primate embryos, but at the same time at least strongly suggest suitability of their process for human embryos. In most societies, however, such procedures are currently deemed not appropriate for use in preparation of human stem cells, as a human embryo would necessarily be terminated. Thus, a dilemma exists in which the potential life-saving benefits of human stem cells is confronted with the fact that heretofore known methods invariably lead to the destruction of a human embryo.

Therefore, while numerous methods and compositions for primary cells, and especially modifiable cells (e.g., by virus or differentiation) are known in the art, improved methods and compositions are needed to overcome the problems associated with current practice.

### SUMMARY OF THE INVENTION

The present invention is directed to compositions and methods of cell culture for primary cells under low-oxygen environment to obtain one or more derivative cells with predetermined and desired characteristics. In one preferred aspect contemplated methods include one step in which a primary mammalian cell is obtained (e.g., via single-cell biopsy, gross tissue removal, or autopsy). In an optional further step, the cell is infected with a pathogen, and in yet another step, the cell is incubated at a reduced oxygen concentration. In particularly preferred methods, the reduced oxygen concentration is effective to (a) improve susceptibility to infection and/or pathogen propagation where the cell is infected with the pathogen, or (b) change or maintain differentiation status of the cell in a predetermined manner.

In other aspects of contemplated methods, the cell is a primary hepatocyte, typically infected with a virus, and most typically a hepatotropic virus. Most typically, the hepatotropic virus is HCV. In such methods, the reduced oxygen concentration is maintained constant at a concentration of between 0.1 vol % to 10 vol %, and the HCV-containing hepatocytes are propagated over at least five generations.

In other aspects of contemplated methods, the cell is a blastomere obtained by a single-cell biopsy of a blastocyst. In such methods, the biopsied blastocyst is further incubated and then implanted into a female, while the cell is expanded into a population of pluripotent or differentiated stem cells. Thus, the cell may be propagated to under conditions to maintain pluripotency of the cell (e.g., oxygen concentration is reduced to a concentration of between 2 vol % and 12 vol %), and/or propagated under conditions to induce differentiation of the cell to a committed lineage (e.g., oxygen concentration is reduced to a concentration of between 0.1 vol % and 8 vol %).

Various objects, features, aspects and advantages of the present invention will become more apparent from the following detailed description of preferred embodiments of the invention.

### DETAILED DESCRIPTION

The inventors have surprisingly discovered that primary cells and tissues, and especially those infected with a pathogen or susceptible to differentiation can be effectively cultured under conditions that more closely resemble in vivo conditions, and/or conditions that induce or modify a stress response. Among other contemplated culture conditions, a reduced oxygen atmosphere has proven especially effective for such culture.

Therefore, in one aspect of the inventive subject matter, a method of incubating a cell is contemplated in which a primary mammalian cell is provided, wherein the cell may be native, already infected with a pathogen, or infected with a pathogen after the cell is obtained. The cell is then incubated under a stress-inducing condition, and most preferably under a reduced oxygen concentration. Typically, the oxygen concentration is reduced to a degree effective to (a) improve at least one of infection and pathogen propagation where the cell is infected with the pathogen, or (b) change or maintain a degree of differentiation of the cell in a predetermined manner.

Thus, and from one perspective, the inventors contemplate culturing cells in vitro under conditions that would either be normal for the cells in vivo (“in-vivo conditions”), or to achieve activation of pathways regulating the expression of stress-specific genes. Such conditions typically enable and/or improve propagation of the cells in vitro as compared to cells grown under non-stressing, atmospheric pressure and other typical laboratory conditions. Similarly, the inventors contemplate that the use of the above in-vivo conditions may also be employed to enable and/or improve propagation of cell pathogens of cells in vitro as compared to cell pathogens propagated under typical laboratory conditions.

With respect to suitable cells, it is contemplated that all eukaryotic cells and cellular structures (including tissue and organ cultures) are appropriate for use herein. For example, contemplated cells include fully differentiated cells (e.g., myocytes, neurons, hepatocytes, etc.), progenitor cells of a specific lineage (e.g., leukoblasts), as well as stem cells and/or embryonic cells (see below). Furthermore, it should be recognized that the cells may be cultivated as a pure cell line, in co-culture with other cells (e.g., for adhesion, nutrition, etc.), or as tissue/organ. Most preferably, contemplated cells are primary cells (i.e., cells that are not immortalized, for example, by viral transformation or other known methods), and are obtained from biopsy and/or surgery. Alternatively, contemplated primary cells may also be obtained from a frozen or otherwise preserved state. Consequently, contemplated cells may have been subjected to prior treatments, including enzymatic treatment (e.g., with collagenase and/or trypsin), mechanical treatment (e.g., centrifugation, shear separation, etc.), and/or chemical treatment (e.g., using one or more solvents). In alternative aspects, numerous cells other than eukaryotic cells and structures are also contemplated, including all types prokaryotic and fungal cells. While culture of mammalian cells is typically preferred, and culture of human cells is even more preferred, it should also be recognized that culture according to the inventive subject matter is not limited to a particular phylogenetic species or family. Therefore, suitable cells may be of vertebrate origin (e.g., mammalian, including human, primate, rodent, etc., or non-mammalian, including fish, birds, etc.) or of invertebrate origin (e.g., insect, including arthropods, lepidoptera, etc.). In yet further contemplated aspects, it should be recognized that while primary cells are preferred, non-primary cells (e.g., immortalized cells or cell lines) are also deemed suitable herein.

Likewise, it should be appreciated that the pathogen may vary considerably. Therefore, contemplated pathogens include viruses, bacteria, fungi, intra- and extracellular parasites, and multi-cellular pathogens. Further especially preferred pathogens especially include those that are at least in part metabolically and/or reproductively coupled to one or more of the host cells enzymatic/genetic components, which most preferably, are correlated with the cells response to a stress event.

It should be especially recognized that at least some of contemplated in-vivo conditions may actually be considered under certain aspects stress conditions in vitro, including hypoxia, heat stress, oxidative stress, UV-irradiation, inflammation, etc., all of which are known to elicit specific stress responses (which may be characterized as comprising expression of a set of specific genes). Such stress responses are considered to enable and/or improve propagation of the cells in vitro, as compared to cells grown under typical laboratory conditions, and/or to enable and/or improve propagation of cell pathogens of cells in vitro as compared to cell pathogens propagated under typical laboratory conditions.

For example, it is generally recognized in the art that hepatocytes in vivo are actually in an environment that is at least somewhat lower in oxygen concentration than other tissues, and most of the currently known in vitro hepatocyte cultures are performed under non-limiting oxygen conditions. However, such in vitro conditions are not expected to accurately reflect the in vivo environment of hepatocytes, and may therefore present an impediment for establishing conditions that allow replication of hepatotropic viruses, or even worse, may lead to misinterpretation of results obtained under non-in vivo-like conditions. Consequently, in a preferred aspect of the inventive subject matter, the inventors contemplate that hypoxic conditions (especially those as described below) may be employed to significantly improve efficacy of a viral infection with a hepatotropic virus (first infection and/or re-infection), and/or viral propagation of such viruses. Thus, the inventors further contemplate that hypoxic conditions will result in significantly increased viral titers.

For example, hypoxic conditions may be employed for immortalized hepatocytes and/or for primary hepatocytes (which may be in co-culture or on a structural support) in an in vitro model of a hepatitis B or hepatitis C infection. However, numerous other viruses, including non-hepatotropic viruses are also contemplated. Exemplary alternative viruses include numerous Flaviviruses, Arboviruses, and the HIV virus. Of course, it should be appreciated that each virus in combination with a particular hepatocyte type (e.g., parenchymal cell, stellar cells, etc.) will have a defined hypoxic condition under which efficacy of infection and/or viral propagation is particularly desirable, and that such conditions can be found by a person of ordinary skill in the art without undue experimentation.

Without wishing to be bound by a specific theory, the inventors contemplate that the improvement in efficacy of infection and/or propagation is at least partially mediated by one or more genes known to be modulated (e.g., up- and/or downregulated) under hypoxic conditions (e.g., IGFBP-1). Consequently, the inventors contemplate that hypoxic conditions may be especially useful in an in vitro model to test and/or screen for antiviral drugs whose action is at least to some degree dependent on the presence of gene products expressed under hypoxic conditions. Furthermore, ex vivo hypoxic conditions may also be used to evaluate efficacy of antineoplastic drugs under an environment that more closely resembles a tumor cell environment. Alternatively, or additionally, contemplated hypoxic conditions may also be employed in vitro to predict success of treatment efforts prior to commencement. For example, a suitable treatment regimen may be established, or a potential non-responder may be identified. Among various drugs expected to be especially effective under hypoxic conditions, IFN may be employed as an antiviral agent.

Still further contemplated uses of hypoxic growth conditions include transformation of hepatocytes with nucleic acids (or analogs thereof), and it is especially contemplated that transformation efficiency is markedly increased. Based on further observations, the inventors also discovered that hypoxic conditions may be employed to generally improve transformation efficiency (e.g., using nucleic acids, viral vectors, viruses, phages, etc.) in numerous cell lines other than hepatocytes. Preferred nucleic acids will include those that encode a polypeptide (e.g., dsDNA or mRNA), but non-coding nucleic acids (e.g., antisense RNA or short interfering RNA) are also contemplated.

In still another aspect of the inventive subject matter, the inventors contemplate that hepatocytes, and particularly primary hepatocytes can be effectively and stably propagated in a hypoxic and controlled environment over at least two, more typically at least five, even more typically at least ten, and most typically at least twenty generations. For example, primary cells are isolated from human liver using procedures well known in the art (see e.g., Gerlach et al. in J. Invest. Surg. (2003) Vol. 16(2), p83-92). Such isolated cells are then propagated in Eagle minimum essential medium with 2 mM L-glutamine and Earle's BSS adjusted to contain 1.5 g/L sodium bicarbonate, 0.1 mM non-essential amino acids, and 1.0 mM sodium pyruvate. The medium is then supplemented to contain 10% fetal bovine serum. Culture temperature is adjusted to 37.0° C., and the oxygen concentration is controlled to a range between 0.1%-8%. Subculturing is typically performed by trypsination and adding fresh culture medium, aspiration, and dispensation into new culture flasks (Split ratio typically 1:3 to 1:6).

Of course, it should be recognized that the liver cells may be derived from sources other than a human liver, and all non-primary sources are contemplated suitable for use in conjunction with the teachings presented herein. For example, where human hepatocytes are preferred, biopsy specimen, cryo-preserved specimen, and previously cultured hepatocytes are particularly preferred. On the other hand, non-human hepatocytes may also be employed, and specifically preferred non-human cells include those isolated from primates, and/or rodents. In yet further contemplated aspects of the inventive subject matter, it should be recognized that hepatocytes may be cultured not only under the conditions provided above, but also in co-culture, as tissue fragments and even hepatic lobes using hypoxic immersion and/or perfusion. With respect to further suitable cell and tissue culture conditions, it is contemplated that protocols well known in the art may be employed or modified without undue expenditure of experimentation. For example, even hyperoxic conditions (i.e., O₂ greater than 21 vol %) may be employed where tissue fragments and tissues are grown.

A particularly useful controlled environment for introducing hypoxic conditions is a modular incubator chamber, such as previously disclosed by Billups and Rothenberg, see e.g. U.S. Pat. No. 3,886,047 to Billups issued May 1975. Such incubators may provide manual and/or automated control of oxygen supply to the cells. Most preferably, contemplated incubators will provide a preset concentration of oxygen to the cell, which may be maintained over the entire period of incubation or which may be gradually or stepwise increased and/or decreased.

With respect to the hypoxic conditions, it is generally contemplated that suitable culture conditions include those in which oxygen concentrations are below normoxic conditions (i.e., below 21%, and most typically below 10%) for hepatocytes. However, it is especially preferred that the oxygen concentration is between 0.1-10%, and more typically between 4-8%, in a continuously controlled atmosphere. Where desired, lower oxygen concentrations include those in which oxygen is present for at least some time in a concentration of about 0.1-8%, and more typically 0.5-4% (in rare cases even lower). For example, where the cell is a hepatocyte infected with the HCV virus, the oxygen concentration may be held constant at a concentration of between 0.1 vol % to 10 vol %, and more preferably between about 3 vol % and 8 vol %. Based on the inventors' experience (data not shown), it is contemplated that such cultured cells can be propagated over at least 3, more typically at least 5, and most typically at least 10 generations.

Furthermore, it should be appreciated that the oxygen concentration may be variable, and it is contemplated that hypoxic concentrations may be temporarily maintained or, where the atmosphere it continuously hypoxic, that the oxygen concentration may fluctuate in a predetermined manner. For example, in one aspect of the inventive subject matter, hypoxic conditions may be generated by oxygen depletion in a pneumatically closed environment up to a predetermined oxygen-depleted value. In another example, hypoxic conditions may be generated by reduction of the oxygen concentration in a gas feed to a pneumatically open environment. Preferred incubators can provide a range of controlled (stable) oxygen environments, and can further control for different ambient atmospheric pressures.

Stable propagation of primary hepatic cells or hepatic tissue using hypoxic conditions is particularly surprising and unexpected as numerous publications indicate that hepatocytes are typically subject to significant metabolic stress at reduced oxygen conditions (see e.g., Tumor Biology (2001), Vol. 22: 310-317). Moreover, the inventors contemplate that hypoxic conditions may also be employed to improve hepatic cell and tissue culture of non-primary hepatocytes and hepatic tissue. Suitable alternative hepatic cells and tissues include immortalized (e.g., viral, oncogenic, or otherwise) hepatocytes, which may or may not be further modified, and hepatic co-cultures.

In another example, suitable cells include blastomeres, and especially single blastomeres obtained by single-cell biopsy from a blastocyst. Typically, and under most conditions, isolated single blastomeres fail to expand to a viable population of pluripotent stem cells. Here, the inventors surprisingly discovered that one or more cells obtained from an IVF embryo biopsy (i.e., blastocyst biopsy) can be used for propagation of such cells into human pluripotent embryonic stem cells. It should be particularly recognized that in such protocols pluripotent human embryonic stem cells can be obtained while at the same time preserving the embryo for implantation (most typically after further incubation, e.g., at reduced oxygen concentration) into a female. Indeed, IVF embryo biopsy has been applied extensively at numerous clinics and has been shown to have no adverse affect on the embryo's potential to implant or develop to a normal, healthy baby. Moreover, based on various studies the success rate for implantation of such biopsied blastocysts is at least encouraging (Fertil Steril. 2003 January; 79(1): 81-90).

With respect to the embryo biopsy, it is generally contemplated that all known manners of biopsy are suitable. Suitable protocols are described, for example, in Fertil. Steril., 2003, 80(2); pages 453-5, Reprod. Biomed. Online, 2003, 6(2): pages 226 et seq., or Prenat. Diagn., 2002, 22(6); pages 525-33. In a typical exemplary procedure, IVF embryo biopsy is performed on day 3 after egg collection. Generally, only embryos that have developed to 5 cells or greater by day 3 are suitable for biopsy. First, the zona pellucida of the day 3 embryo is perforated with a laser beam (or acid), and 1-2 cells are removed from the embryo (typically via micromanipulator and micropipette). After biopsy, the embryos are returned from the process medium to the culture medium and cultured to a later stage suitable for implantation (typically day 4, or blastocyst stage).

Where more than one embryo is biopsied, it should be recognized that non-reimplanted embryos may be frozen using cryopreservation techniques well known in the art. During cryopreservation, the culture medium of the embryo is typically admixed with a cryoprotectant, which may be permeating (e.g., propanediol), or extracellular (e.g., sucrose, or lipoprotein). Depending on the timing, it should be recognized that the embryos can be frozen at any stage after biopsy up to and including the blastocyst stage (5-7 days after fertilization).

With respect to the removed human embryonic cell, it is generally contemplated that all manners of propagation suitable for non-human embryonic stem cells are deemed appropriate for use herein (see e.g., Biology Of Reproduction 68, 2150-2156 (2003), Biology Of Reproduction 69, 2007-2014 (2003), or Stem Cells, 21, 546-556 (2003)). For example, the biopsied cell may be plated on mammalian inactivated embryonic fibroblasts. After a suitable incubation period (typically between 7-21 days), cell masses derived from the biopsied cell are removed from the support layer and gently trypsinated to dissociate. The dissociated cells are re-plated on embryonic feeder layers in fresh medium, and observed for colony formation. Colonies demonstrating stem cell-like morphology (i.e., compact colonies having a high nucleus to cytoplasm ratio and prominent nucleoli) are individually selected, and split again as described above. Resulting human embryonic stem cells are then routinely split by brief trypsinization or exposure to Dulbecco's Phosphate Buffered Saline (without calcium or magnesium and with 2 mM EDTA) every 1-2 weeks as appropriate. Alternatively, or additionally, early passage cells may be frozen and stored in liquid nitrogen. Alternatively, feeder layer free culture may also be performed as known in the art.

Thus, low-oxygen conditions (e.g., between 0.2 vol % and 16 vol %., and more preferably between 2 vol % and 12 vol %) may be employed to expand the stem cells while maintaining the pluripotent character of the stem cells. On the other hand, and especially where desired, differentiating factors (e.g., cytokines) may be added to such cultivated cells. Alternatively, the oxygen concentration may be further lowered (e.g., to a concentration between 0.05 vol % and 15 vol %., and more preferably 0.1 vol % and 8 vol %.) to induce differentiation of the cell from the pluripotent stage to a committed lineage (e.g., hematopoietic, neurogenic, hepatogenic, etc.). With respect to the reduced oxygen concentration for culture of so obtained biopsied cells and stem cells, it should be recognized that different types of stems cells will have different requirements.

Thus, specific embodiments and applications of oxygen-controlled environments for cell- and tissue culture have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive mianner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. A method of incubating a cell, comprising
obtaining a primary mammalian cell;
optionally infecting the cell with a pathogen; and
incubating the cell at a reduced oxygen concentration, wherein the oxygen concentration is reduced to a degree effective to (a) improve at least one of infection and pathogen propagation where the cell is infected with the pathogen, or (b) change or maintain a degree of differentiation of the cell in a predetermined manner.

2. The method of claim 1 wherein the cell is a primary hepatocyte.

3. The method of claim 2, wherein the cell is infected with the pathogen, and wherein the pathogen is a hepatotropic virus.

4. The method of claim 3 wherein the hepatotropic virus is HCV.

5. The method of claim 4 wherein the reduced oxygen concentration is maintained constant at a concentration of between 0.1 vol % to 10 vol %.

6. The method of claim 4 wherein the HCV-infected cell is propagated over at least 5 generations.

7. The method of claim 1 wherein the cell is infected with a virus.

8. The method of claim 1 wherein the cell is a blastomere obtained by single-cell biopsy of a blastocyst.

9. The method of claim 8 further comprising a step of further incubating the biopsied blastocyst, and a step of implanting the further incubated blastocyst into a female.

10. The method of claim 9 wherein the cell is propagated under a condition to maintain pluripotency of the cell.

11. The method of claim 10 wherein the oxygen concentration is reduced to a concentration of between 2 vol % and 12 vol %.

12. The method of claim 9 wherein the cell is propagated under a condition to induce differentiation of the cell to a committed lineage.

13. The method of claim 12 wherein the oxygen concentration is reduced to a concentration of between 0.1 vol % and 8 vol %.
